(19) United States
(12) Patent Application Publication
(10) Pub. No.: US 2011/0033546 A1
(43) Pub. Date: Feb. 10, 2011

(54) Pure sustained dichroa ferbrifuga alkone formulation

(76) Inventor: Xiao Qi Wang, Ningbo (CN)

Correspondence Address: Wang Xiao Qi, 22 Sunrise Bay Blvd., Tuckerton, NJ, 08087, US

(21) Appl. No.: 12/462,770

(22) Filed: Aug. 10, 2009

(51) Int. Cl. A61K 9/14; A61K 36/18; B05D 3/00; A61P 35/00

(52) U.S. Cl. 424/494; 424/725; 427/2.14

(57) ABSTRACT

Now is provided a new sustained release drug preparation comprising such an inclusion complex of a medical compound with dichroa febrifuga alkone derivative (DFAD), which sustains or retards the dissolution and release of the DFAD at a controlled rate from the inclusion complex and hence from the drug preparation containing the DFAD, so as to maintain the concentration of the DFAD in blood at an effective level for prolonged time.

### BACKGROUND OF THE INVENTION

The product is a formulation of pure sustained release oral dosage micropellets in a capsule which contains dichroa febrifuga alkone derivative (DFAD) when taken by a patient and are comprised of inner seeds coated with DFAD. The oral dosage formulation is administered by separating the upper and lower parts of the capsule and placing the micropellets on drug. A delay in absorption was found and the bioavailability was reduced significantly.

There is thus a need for a slow-release DFAD composition which provides satisfactory bioavailability and absorption pattern when taken orally with drug.

The oral sustained release DFAD formulation of the present invention provides a means to administer DFAD in a micropellet formulation which enables patients to receive the correct therapeutic blood level of DFAD.

The micropellets of this invention are utilized in an easily openable capsule containing a sufficient amount of micropellets to provide a dosage unit of DFAD. The dosage unit administered to a patient is determined by the age, size and condition of the patient as well as the severity of the disease.

### DESCRIPTION OF THE PRIOR ART

The natural source of DFAD is abounding. DFAD is extracted from plant named dichroa febrifuga (Blue Evergreen Hydrangea). The plant has 4 to 8 inch long dark green resemble the foliage of Hydrangea with prominent veins and small serrations. The terminal end of the branches hold clusters of Hydrangea-like flowers with white buds opening to bright blue flowers in spring and summer that are followed by metallic blue berries. As with the blue forms of Hydrangea the shade of blue of the flower is determined by soil pH and more acid soils produce bluer flowers. Plant in part sun to light shade with moderately moist soil. It is hardy and evergreen to 20-25 degrees F. but defoliates such below these temperatures but plants knocked back by cold resprout from hard wood. Dichroa febrifuga is native to Nepal eastwards to southern China and into south-east Asia, where it grows at the forest edge. The specific epithet febrifuga is in reference to the use of the plant as a febrifuge, acting to reduce fever. Its use as such is reported in China.

Febrifugine was isolated from plant and synthesis. For example, febrifugines is isolated cis-febrifugine and trans-febrifugine from Hydrangea macrophylla (Saxifragaceae); these compounds had already been isolated from Dichroa febrifuga (antimalarial natural drug) by Koepfli et al. in 1947 and from Hydrangea umbellate by Ablondi et al. in 1952. Synthesis of febrifugins was first achieved in 1952 by Baker et al., who later reported that febrifugine obtained from D. febrifuga corresponds to the synthetic cis-febrifugine, whereas isofebrifugine corresponds to the synthetic trans-febrifugine. Afterwards, Barringer et al. established through detailed H-NMR analysis that the assignments by Baker et al. should be reversed, i.e., febrifugine has a trans-orientation and isofebrifugine has a cis-configuration. Thus, the absolute configurations of febrifugines were established.

So far, it has no report regarding a sustained release preparation of DFAD.

### DETAILED DESCRIPTION OF THE INVENTION

A sustained release preparation may be a good thing for patients. It's easier to take a medication once a day than twice a day—and a sustained release version may insure a more steady blood level of the medication.

Febrifugine and isofebrifugine derived from Chinese hydrangea are known to have strong activities against tropical malarial protozoan. The chemical structures of febrifugine and isofebrifugine are known to show such strong activities against malarial. The activity of these febrifugine compounds have been known from old times as active ingredients of Chinese medicines such as “JOSAN”.

Malaria is one which of most serious infectious diseases. In the known infectious disease, it is only inferior to human health's harm to pulmonary tuberculosis. According to the World Health Organization reported that world has every 3 to 600,000,000 people infects malaria, dies the approximately 3.000,000 people. In 2005 the 58^{th} World Health Assembly pointed out: every year malaria continues the death which creates more than 100 ten thousand may prevent, particularly in Africa's babies and other frail crowds, and this disease continues to threaten the Americans, Asians and the Pacific section several million person of lives. Although the existing medicine (for example quinoline chloroquinoline and so on) has certain curative effect to malaria, but the human body will have the drug resistance rapidly in the course of treatment. As early as in ancient China used the saxifragaceae Chinerse herb dichroa febrifuga to use in the malaria shot.

The following specific examples will provide detailed illustration of methods of producing DFAD according to the present invention and pharmaceutical dosage units containing DFAD. Moreover, examples will be given by way of pharmaceutical testing performed with DFAD to demonstrate its effectiveness. These examples are not intended, however, to limit or restrict the scope of the invention in any way, and should not be construed as providing conditions, parameters, reagents, or starting materials which must be utilized exclusively in order to practice the present invention.

### EXAMPLE 1

1 kg polyvinylpyrrolidone (PPD) (molecular weight 40.000) were dissolved in 10 liter of isopropand, and 1 kg of micronized DFAD were dispersed in there. 3.5 kg of sugar was placed in suspension and mix. The DFAD is coated onto the sugar seed by first combining it with a water soluble system such as polyethylene glycol or polyvinylpyrrolidone.

The resulting DFAD coated sugar seeds are then coated with a pharmaceutically acceptable waterinsoluble system such as ethylcellulose, cellulose acetate butyrate or cellulose triacetate, with ethyl cellulose preferred. This coating enables release of the DFAD. The average diameter of each of the finished micropellets is about 0.4 to 0.6 mm, preferably about 0.5 mm. This provides a coating with a sufficient amount of channels to enable the DFAD to be released.

The dissolution rate depends on the weight of the micropellets and solvent system.

The pellets were screened.

As desired, the final coated products containing an ethylcellulose coating level of 1% was prepared. The pellets were dried under vacuum.

The products contained 99.0% by weight of DFAD and 1% by weight ethylcellulose coating.

### EXAMPLE 2

Plasma concentration of DFAD in rat was determined by regular methods.

Plasma Concentrating of DFAD

| | Regular preparation of DFAD | Sustained release of DFAD |
|---|---|---|
| 1 h | 75 mg/ml | 60 mg/ml |
| 2 h | 68 mg/ml | 55 mg/ml |
| 4 h | 52 mg/ml | 45 mg/ml |
| 8 h | 42 mg/ml | 38 mg/ml |
| 24 h | 21 mg/ml | 28 mg/ml |
| 48 h | 15 mg/ml | 20 mg/ml |
| 72 h | 8 mg/ml | 16 mg/ml |

The data in table shows that plasma concentration of DFAD in sustained preparation was not significantly different from DFAD in regular preparation before 8 hours. But it did after 8 hours. The data shows that bioavailability of sustained release preparation of DFAD is better than regular preparation of DFAD.

### EXAMPLE 3

The formation of an inclusion complex of a medical compound with DFAD in accordance with the process described above was confirmed by various methods such as powder X-ray diffraction, dissolution behavior, scanning electron microscope analysis, differential thermal analysis (DTA) and infrared absorption (IR). Inclusion complexes were prepared using DFAD as a medical compound, and the behavior of dissolution and release of DFAD from the inclusion complex in the capsule form, as well as the behavior of dissolution and release of DFAD from compressed capsule containing the inclusion complex were determined.

The characteristic peaks of the individual components have disappeared, but instead, such a diffraction pattern which is different from the diffraction patterns of a physical mixture of both the components has been given. These results of the X-ray diffraction patterns support the fact that DFAD and a pharmaceutical acceptable have complex with each other and formed an inclusion complex of them having a structure different from the original structures of the individual components.

### EXAMPLE 4

To demonstrate the behaviors of dissolution and release of the medical compound from the sustained release pharmaceutical composition according to this particular embodiment of this invention, the following tests were conducted. Thus, an inclusion complex of DFAD shows a highly controlled release rate of the tablet samples. The respective tablet samples were separately placed into water and release into water from the tablet was determined with lapse of time which exhibits a similar variation in the amount of DFAD as dissolved and released from a tablet sample. It is observed that the DFAD was absorbed promptly into the blood and disappeared quickly from the blood when the original tablet which is mixture of DFAD with starch in the compressed tablet form was orally given, and that in contrast, pure sustained release of DFAD was maintained in the blood at substantially steady concentrations for prolonged period of time.

### EXAMPLE 5

The novelty of the present invention resides in the mixture of the active ingredients in the specified proportions to produce DFAD and in the preparation of dosage units in pharmaceutically acceptable dosage form. The tern “pharmaceutical acceptable dosage form” as used hereinabove includes any suitable vehicle for the administration of medications known in the pharmaceutical art, including, by way of example, tablets, capsules, syrups, and elixirs with specified ranges of DFAD concentration.

What is claimed as new and desired to be protected by letter patent is set forth in the appended claims:

1. A pure sustained release of dichroa ferbrifuga alkone derivative (DFAD). Composition comprising DFAD—containing micropellets having an surface thereof a coating of a pharmaceutically acceptable agent—thylcellulose in a micropellets.

2. The sustained release drug preparation as claimed in claim 1, wherein the DFAD is derivative of dichroa febrifuga alkone.

3. The sustained release drug preparation as claimed in claim 1 wherein comprising further a pharmaceutically acceptable carrier for said inclusion complex.

4. The Pure Sustained Release Dichroa Ferbrifuga Alkone Formulation comprising: DFAD 99% by weight and ethylcellulose 1% by weight.

5. DFAD, according to claim 4 wherein said dichroa febrifuga alkone is extracted from plant named dichroa febrifuga (blue evergreen hydrangea) and hydrangea umbellate.

6. A process for producing DFAD comprising:
(a) 1 KG of micronized DFAD were dispersed in 3.5 KG of sugar was placed in suspension and mix;
(b) The DFAD is coated onto the sugar seed;
(c) The resulting DFAD coated sugar seeds are then coated with a pharmaceutically acceptable waterinsoluble system such as ethylcellulose, cellulose acetate butyrate or cellulose triacetate, with ethyl cellulose preferred;
(d) This coating enables release of the DFAD. The average diameter of each of the finished micropellets is about 0.4 to 0.6 mm, preferably about 0.5 mm;
(e) This provides a coating with a sufficient amount of channels to enable the DFAD to be released;
(f) The pellets were screened; and
(g) The final coated products containing an ethylcellulose coating level of 1% was prepared. The pellets were dried under vacuum.
